(19) United States
(12) Patent Application Publication
(10) Pub. No.: US 2011/0061663 A1
(43) Pub. Date: Mar. 17, 2011

(54) Infant Sleep System

(75) Inventor: Wendy Hauser, Lehighton, PA (US)

(73) Assignee: BEAUTIFUL IDEAS CORPORATION, Jupiter, FL (US)

(21) Appl. No.: 12/558,540

(22) Filed: Sep. 13, 2009

(51) Int. Cl. A61F 5/37

(52) U.S. Cl. 128/870

(57) ABSTRACT

The invention provides an infant sleep system that can deter, reduce or eliminate several risk factors affecting infants while sleeping, including, but not limited to SIDS (Sudden Infant Death Syndrome), skull flattening, toxic gas theory, acid reflux or Gastro-Esophageal Reflux (GER), colic, and injuries resulting from falls.

### TECHNICAL FIELD

This invention relates in general to sleeping devices designed for infants, and in particular to an improved sleeping device which eliminates or reduces many common ailments peculiar to infants.

### BACKGROUND ART

Many types and styles of sleeping devices exist for infants. For each of these, there exist both positive and negative aspects, some of which are dependent upon the age and/or size of the infant. Additionally, many additional devices have been proposed to alleviate certain ailments, but these additional devices often are size-specific or are unable to be easily transferred to other devices. Thus there exists a need in the art for a universal, portable system that can be easily resized to fit multiple types, styles and sizes of sleeping devices, and which can eliminate or reduce the impact or severity of several common ailments peculiar to infants.

### DISCLOSURE OF THE INVENTION

The invention comprises several general aspects. Each of those can if desired be combined with additional features, including features disclosed and/or not disclosed herein, the resultant combinations representing more detailed optional embodiments of these aspects.

A first aspect of the invention is a portable, resizable infant sleep system comprising a main section, a lower section, a primary infant securing device, and securing mechanisms; where said main section is secured to a surrounding structure in an angled, elevated orientation via at least a portion of said securing mechanisms; said lower section is secured via at least a portion of said securing mechanisms such that at least a portion of said lower section is in contact with a horizontal plane of said surrounding structure; and wherein an infant may be secured via the infant's torso via said infant securing device such that said infant's head is maintained in an angled, elevated position while at least a portion of said infant's lower half remains in contact with said horizontal plane through said lower section. A parent can alter the angle and elevation of the main section by altering the length and/or tie down location(s) of the securing mechanisms. Additionally, the feet can be elevated separately, leaving only the infants buttocks in contact with the horizontal plane of the surrounding structure

In various embodiments of this aspect the primary infant securing device may comprise a central section, and left and right adjustable side sections; wherein the outer side of said side sections is anchored to said main section, and wherein at least a portion of the inside sections of said adjustable side sections overlap and can be releasably secured together; and wherein said central section is secured via its base to said main section, and its upper portion is releasably secured to said main section. In related embodiments, the system may also comprise a secondary infant securing device located above said primary securing device, and wherein said secondary securing device comprises left and right adjustable side sections; wherein the outer side of said side sections is anchored to said main section, and wherein at least a portion of the inside sections of said adjustable side sections overlap and can be releasably secured together.

In various other embodiments, the system may comprise a skull flattening prevention/correction device. This device may be removably attached, so that it can be properly positioned for maximum effect, or may be folded down underneath the primary infant securing device when not in use. This device has a main section which is used to slightly elevate the neck, allowing the base of the skull to assume its natural curve, and may further comprise side walls with which to restrain the infant's ability to turn their head to one side or the other.

In yet other embodiments, the system may comprise four or more securing mechanisms through which the system may be removably secured to a crib, to a portable, expandable frame, etc. In various forms, the sizes of the individual members may be non-uniform, that is, the member may have a width that is uniform or that varies along its length. Likewise the width of the line of contact between various members and the system may be different or may be the same. Additionally, the number of members, and the locations where they contact the system may differ in different configurations. However, in most embodiments, there are at least two members connecting the main section of the system to its surroundings, and at least two members connecting the lower section to its surroundings.

### ADVANTAGES OF THE INVENTION

The following discussion of advantages is not intended to limit the scope of the invention, nor to suggest that every form of the invention will have all of the following advantages. As will be seen from the remainder of this disclosure, the present invention provides a variety of features. These can be used in different combinations. The different combinations are referred to as embodiments. Most embodiments will not include all of the disclosed features. Some simple embodiments can include a very limited selection of these features. Those embodiments may have only one or a few of the advantages described below. Other preferred embodiments will combine more of these features, and will reflect more of the following advantages. Particularly preferred embodiments, that incorporate many of these features, will have most if not all of these advantages. Moreover, additional advantages, not disclosed herein, that are inherent in certain embodiments of the invention, will become apparent to those who practice or carefully consider the invention.

The foregoing and other objects of the invention are achieved by the system and apparatus described herein which overcome problems isolated, discrete devices targeting a single ailment common to children. These problems include, for example, such things as single use, single position, lack of transportability, child size limitations, and crib size limitations.

Thus, when compared to other sleeping systems, the new infant sleeping system offers several new and important advantages. The advantages offered by the various embodiments of this invention include:
Portability—the device is not restricted in size or shape, and can be used in a wide variety of style and shape of infant cribs. Additionally, the device can be fashioned for use with its own portable, expandable frame, and thus is not limited for use in a crib.
Reduced risk of SIDS—between 3 and 4 months of age, infants begin to roll over from the back position to their sides or, even worse, their bellies. According to the SIDS network website, http://www.sids-network.org, “babies should be encouraged to sleep on their backs during as much of the first year as possible.”
Reduces/corrects skull flattening—when an infant's skull is fatter on one side than the other, getting pressure off that spot by re-positioning is a known way to help gradually reshape the head. By tightening and loosening the straps on the securing mechanisms, the present invention can be repositioned easily, allowing a parent or care giver to prevent, reduce, or correct skull flattening in most cases.
Reduces toxic gas proximity—the present invention address the dangers associated with the release of any harmful gases which might be emitted by a mattress or pad. According to Toxic Gas Theory, most crib mattresses are made with fire retardant chemicals. These chemicals may contain Antimony, Phosphorous, and even Arsenic. When consumed are picked up my common micro-organisms that live in mattresses, such as dust mites, these chemicals can form toxic gases. Since infants are small, they breathe air closes to the mattress surface, were the greatest concentration of these gases exists. By elevating the infant's head, the present invention removes or reduces this possible danger.
Reduces acid reflux—the unique features of the present invention, particularly the ability to elevate the torso of the infant, reduces or eliminates acid reflux (both GER and GERD), and the symptoms associated with it. The present invention allows the infant to sleep in a slightly upright angle, allowing gravity to keep the stomach acid and food where it belongs—in the stomach. By minimizing acid reflux, infants can have a more restful sleep.
Reduces colic—many studies have found that infants sleep best when they are snuggled as if they are being held and carried in their parents arms. The instant invention, suspended by its securing mechanisms, mimics this ability, by simultaneously securing and snuggling the infant, and allowing the infant's motions to slightly sway or rock the invention.
Reduces/eliminates falling danger—as infants get older, one of the biggest fears a parent may have is that their infant will attempt to climb out of their crib and injure themselves in a fall. The present invention removes those fears by keeping the infant firmly secured through the primary and/or secondary infant securing devices.

### BRIEF DESCRIPTION OF THE DRAWINGS

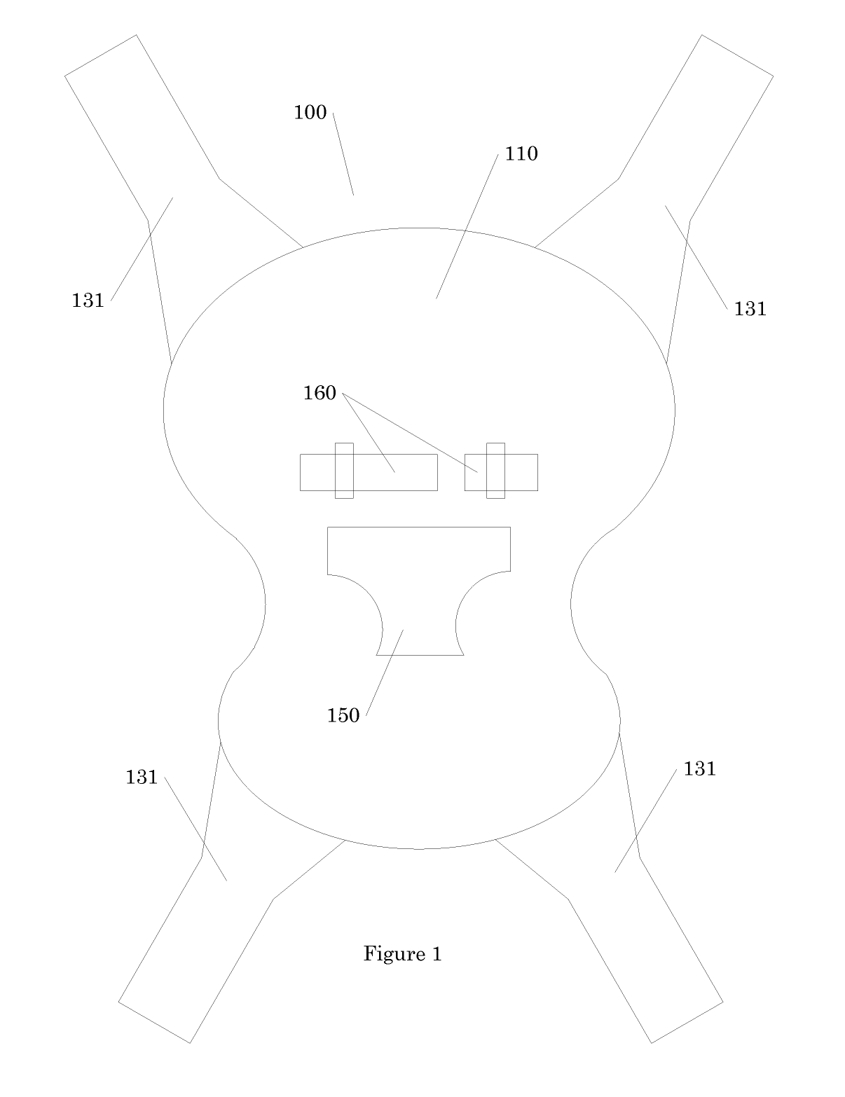

### FIGS. 1-4

FIG. 1 through 4 demonstrate various numbers securing mechanisms 130 used to secure the main 110 and lower 120 sections of the infant sleeping system 100. As is readily apparent from simple inspection, changes in the sizes and shapes for the securing mechanism members 131 result in broad discretion in configuring where said members need to attach to said main and lower sections, and how many are needed. In these figures, the primary and secondary infant securing devices are not shown.

FIG. 1 shows a “wide” embodiment of said members 131, where the line of contact between the members 131 and the main 110 and lower 120 sections is relatively large. In this instance, only two members 131 are used to secure the main and lower sections to the surrounding structure 140.

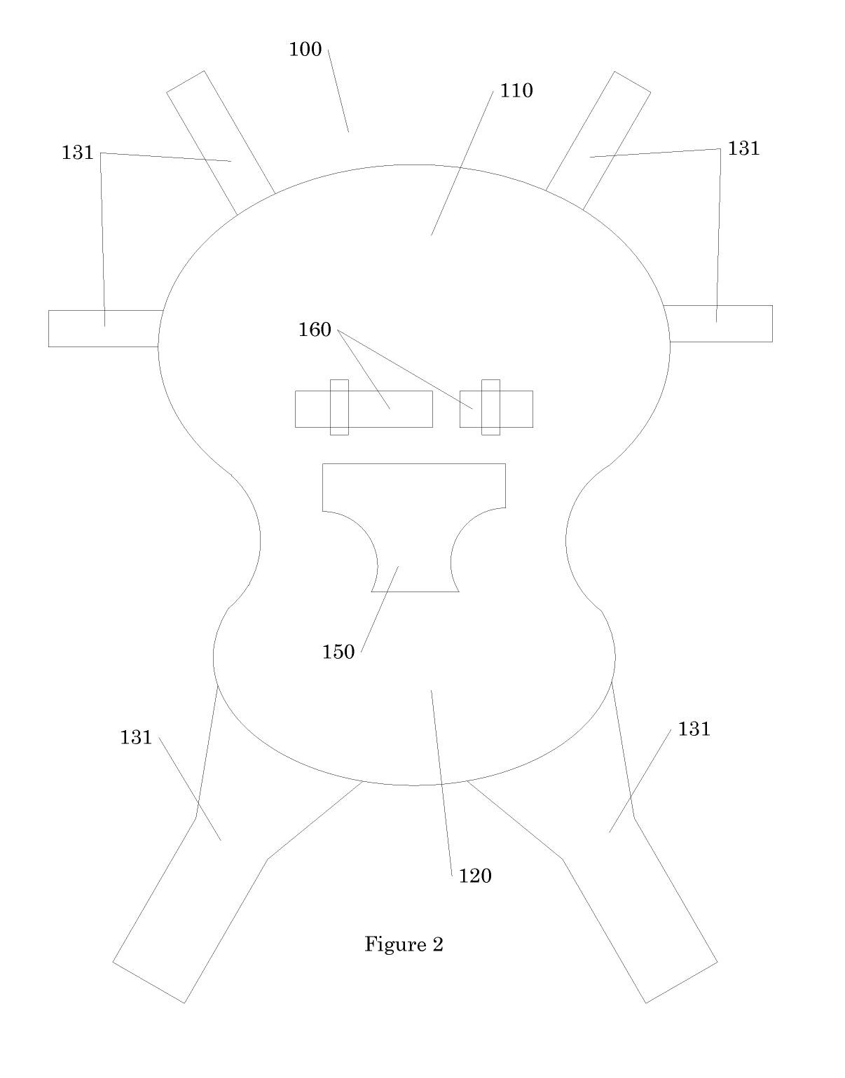

FIG. 2 shows a combination of wide and narrow embodiments of said members 131, wherein the wide embodiments are attached to the lower 120 section, and a greater number of narrower members 131 are used to secure the main 110 section.

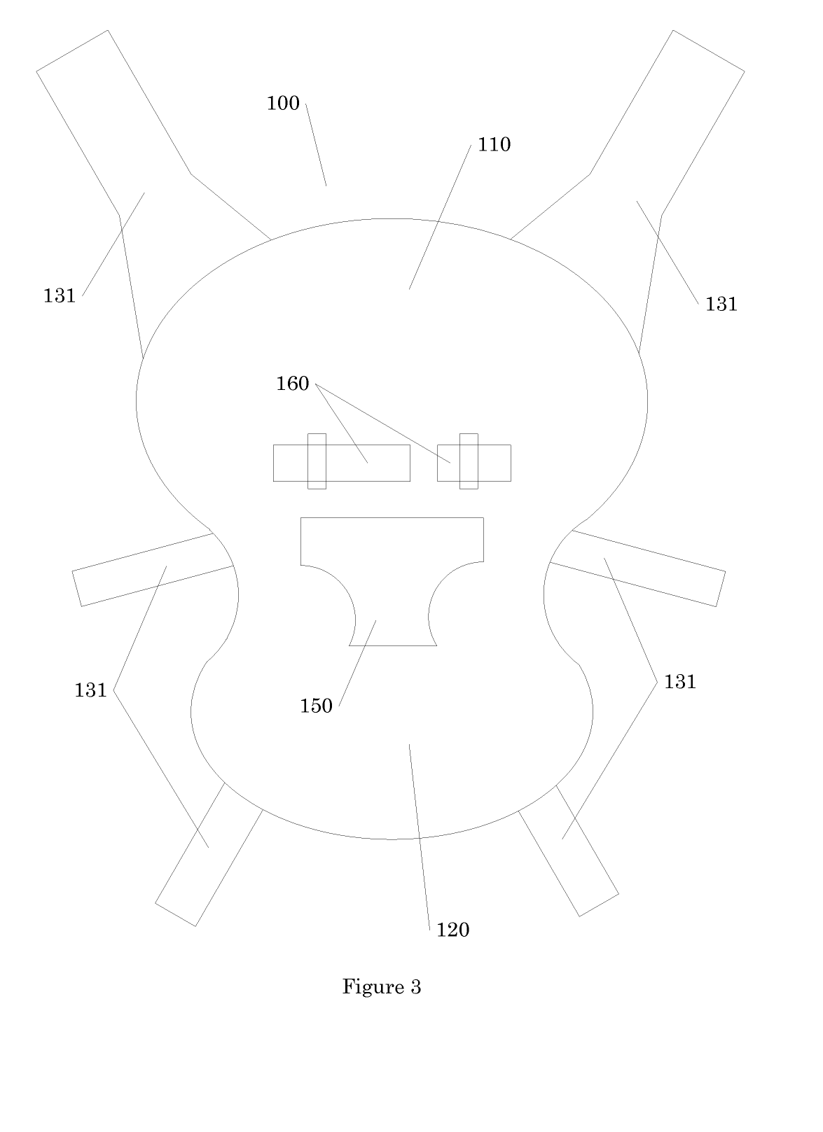

FIG. 3 shows a combination of six members 131 used to secure the system 100 to the surrounding structure 140.

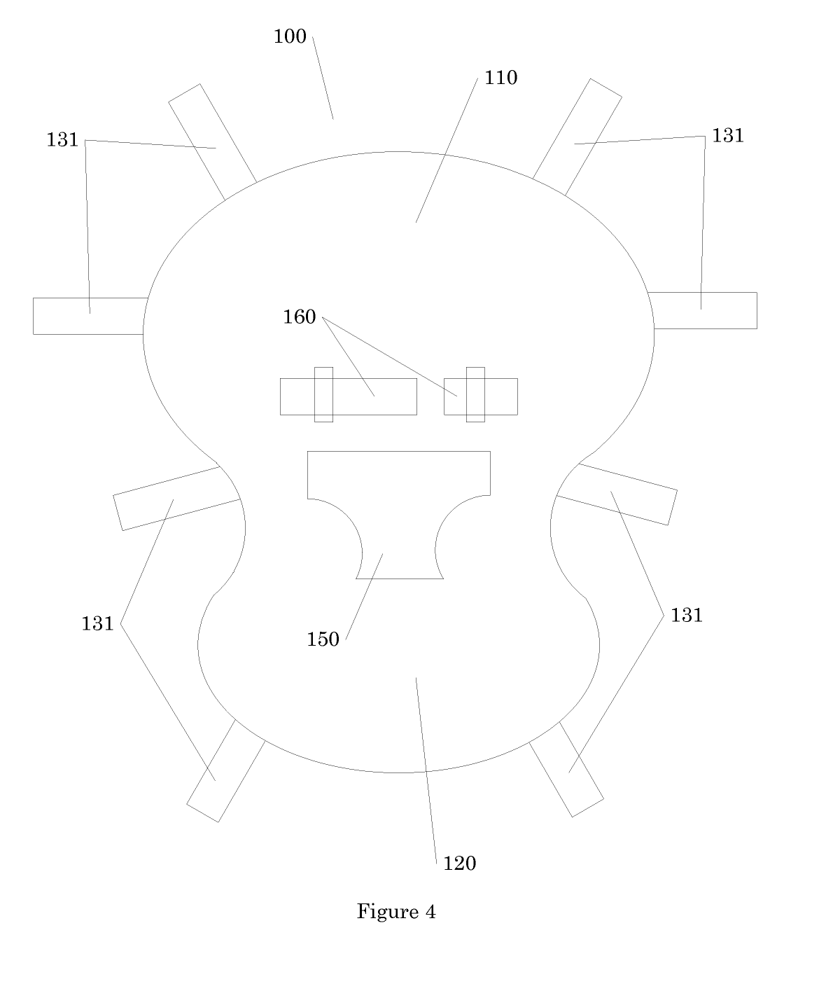

FIG. 4 shows a combination of eight members 131 used to secure the system 100 to the surrounding structure 140.

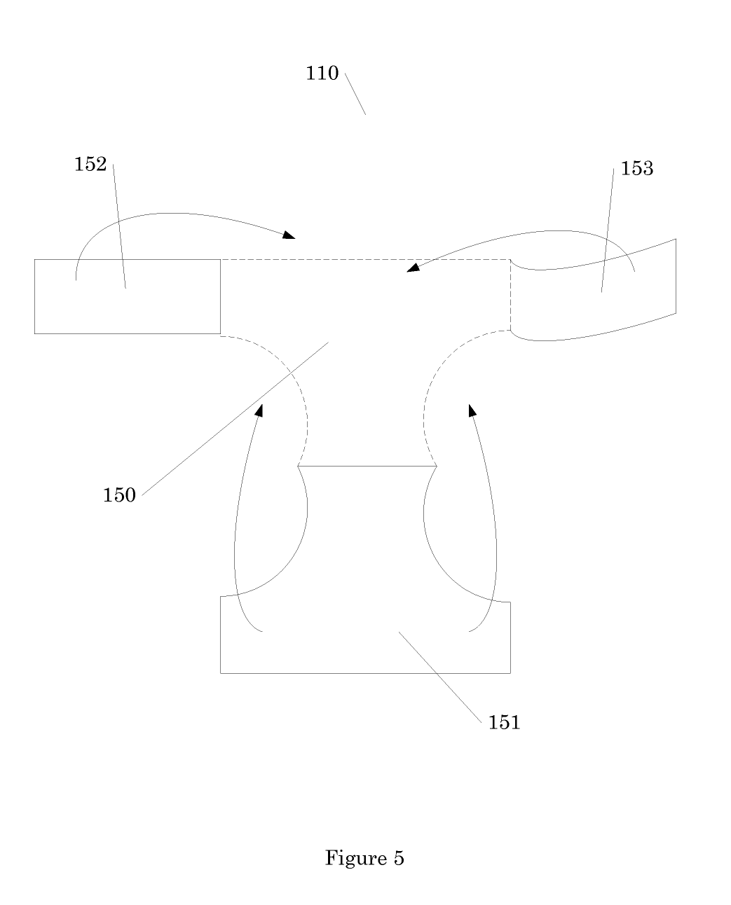

### FIGS. 5-8

FIGS. 5 through 8 demonstrate various configurations for attaching the primary 150 and secondary 160 infant securing devices, and show both the secured and unsecured positions of their component parts. In these figures, the main section 110 is the background.

In FIG. 5, the central section 151, and the left 152 and right 153 side sections are shown with each section full extended outbound, away from their respective releasably secured contact points.

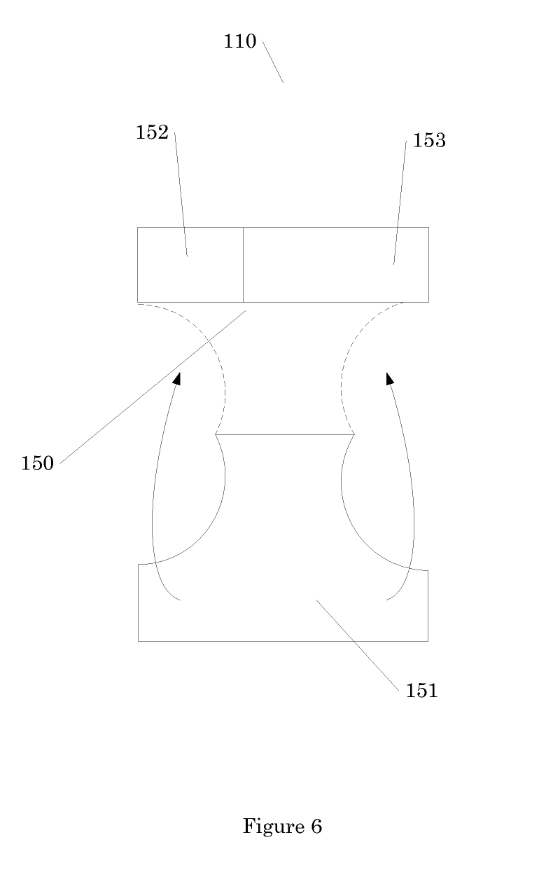

FIG. 6 shows the left 152 and right 153 side sections secured to one another along the approximate center line of the main section 110. The line of attachment for the left and right sections will move left and right from the center line dependant on the size of the infant.

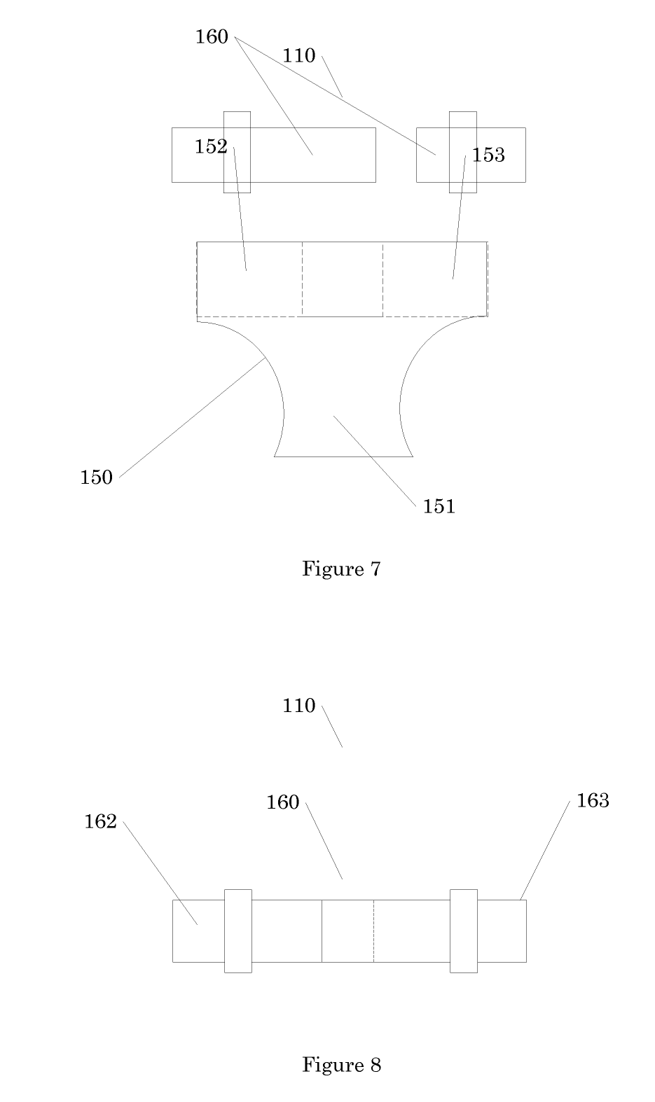

FIG. 7 shows the center section 151 in its secure position, wherein it overlays the left 152 and right 153 side sections.

FIG. 8 shows the secondary infant securing device 160, itself comprising of a left 162 and right 163 side sections, which can be secured in a fashion similar to the primary infant securing device's side sections.

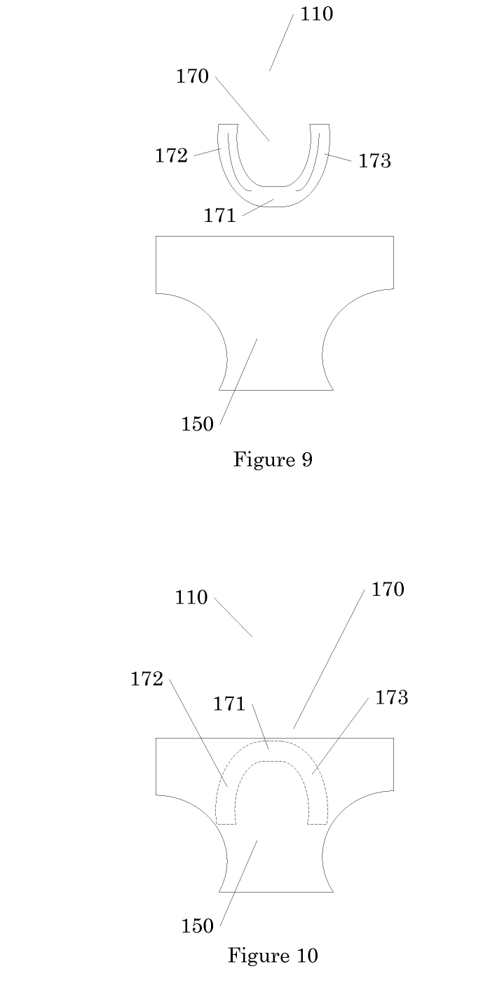

### FIGS. 9-10

FIGS. 9 through 10 show the various potential positions of the skull flattening prevention device 170. The device comprises to side sections 172 and 173, which may be removably secured together, and a central section 171 which slightly elevates the neck, allowing the skull to resist being flattened. The central section may also comprise raised side sections such that the infant's head will not unduly rotate left or right.

In FIG. 9, the device 170 is removably attachable to said main section. When not in place, its anchor points may be covered to provide a smooth, continuous surface.

In FIG. 10, the device 170 allows the central section 171 to be folded downward underneath the primary infant securing device's left 152 and right 153 sections.

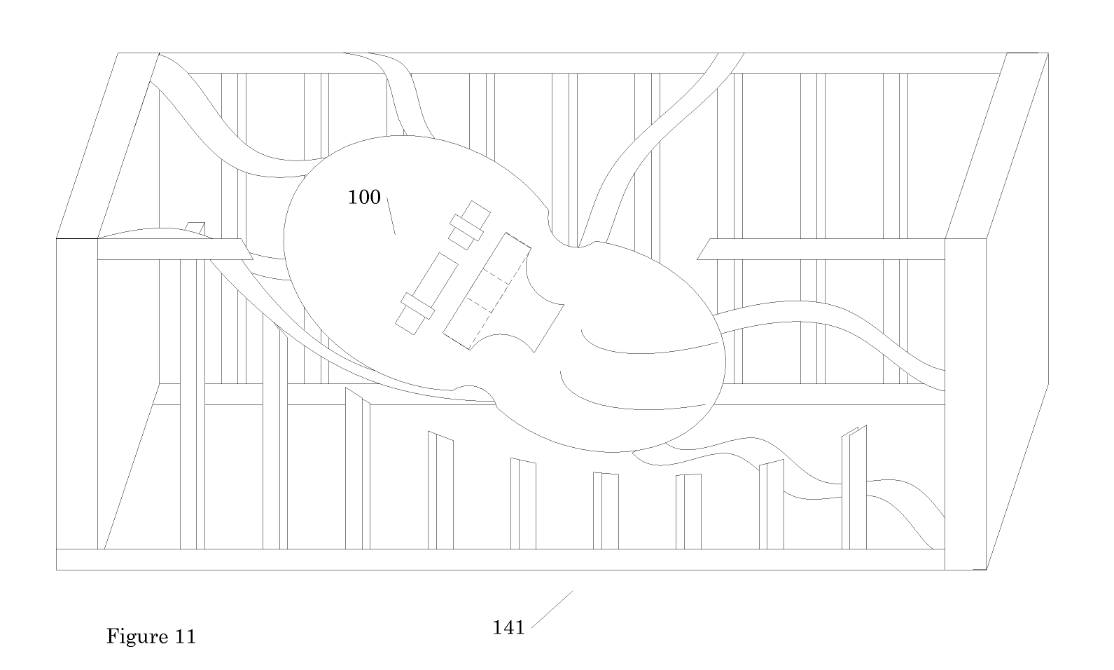

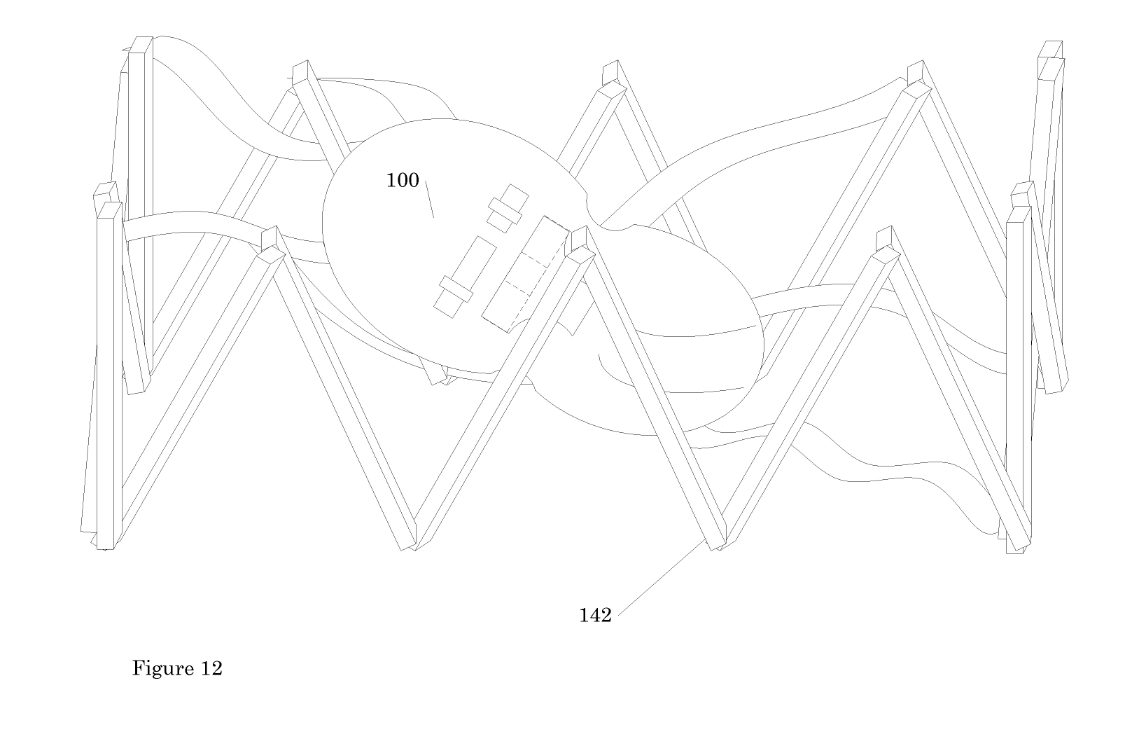

### FIG. 11-12

FIGS. 11 and 12 show a preferred embodiment of the infant sleeping system 100 attached to a crib 141 (FIG. 11) with portions of the crib's side walls removed for clarity, or attached to a portable expandable frame 142 (FIG. 12).

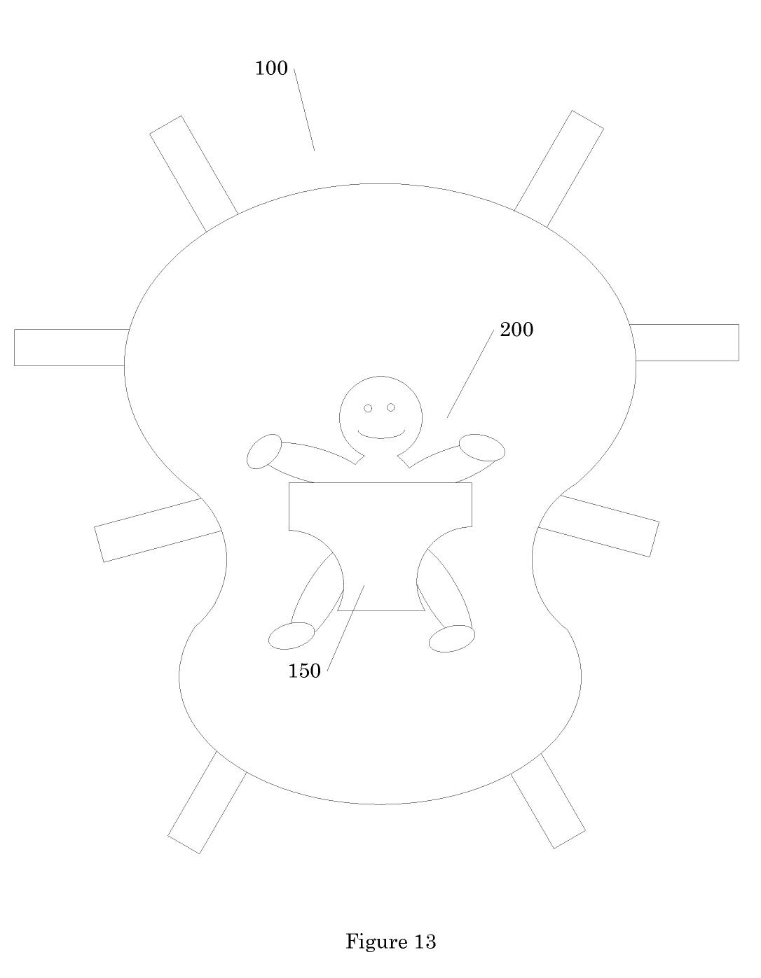

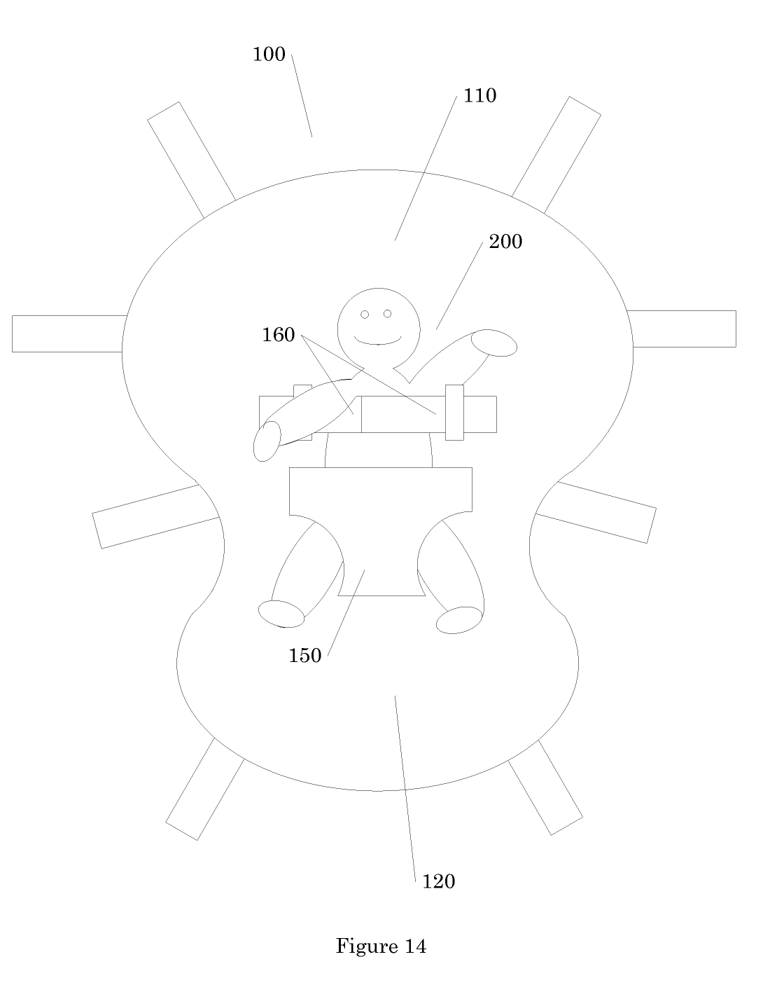

### FIG. 13-14

FIGS. 13 and 14 are top views of the infant sleep system 100 securing an infant 200. In FIG. 13, a small infant is shown; in FIG. 14, a larger infant in shown, necessitating a need for secondary infant securing device 160.

### OBJECT IDENTIFICATION NUMBERS

The following table identifies the objects labeled in the included drawings

TABLE 1

| | |
|---|---|
| 100 | Infant sleeping system |
| 110 | Main section |
| 120 | Lower section |
| 130 | Securing mechanism |
| 131 | Securing mechanism members |
| 140 | Surrounding structure |
| 141 | Crib |
| 142 | Portable expandable frame |
| 150 | Primary infant securing device (PISD) |
| 151 | PISD central section |
| 152 | PISD left section |
| 153 | PISD right section |
| 160 | Secondary infant securing device (SISD) |
| 162 | SISD left section |
| 163 | SISD right section |
| 170 | Skull flattening prevention device (SFPD) |
| 171 | SFPD central section |
| 172 | SFPD left section |
| 173 | SFPD right section |
| 200 | Infant |

1. An infant sleep system comprising a main section, a lower section, a primary infant securing device, and securing mechanisms; where said main section is secured to a surround structure in an angled, elevated orientation via at least a portion of said securing mechanisms; said lower section is secured via at least a portion of said securing mechanisms such that at least a portion of said lower section is in contact with a horizontal plane of said surrounding structure; and wherein an infant may be secured via the infant's torso via said infant securing device such that said infant's head is maintained in an angled, elevated position while at least a portion of said infant's lower half remains in contact with said horizontal plane through said lower section.

2. A system as in claim 1 wherein said primary infant securing device comprises a central section, and left and right adjustable side sections; wherein the outer side of said side sections is anchored to said main section, and wherein at least a portion of the inside sections of said adjustable side sections overlap and can be releasably secured together; and wherein said central section is secured via its base to said main section, and its upper portion is releasably secured to said main section.

3. A system as in claim 1 wherein said main section further comprises a secondary infant securing device located above said primary securing device, and wherein said secondary securing device comprises left and right adjustable side sections; wherein the outer side of said side sections is anchored to said main section, and wherein at least a portion of the inside sections of said adjustable side sections overlap and can be releasably secured together.

4. A system as in claim 1 wherein said main section further comprises a skull flattening prevention device.

5. A system as in claim 4 wherein said skull flattening prevention device is removably attachable to said main section.

6. A system as in claim 4 wherein said skull flattening prevention device is folded such that it is concealed within said primary infant securing device.

7. A system as in claim 1 wherein said portion of said securing mechanisms used to secure said main section comprises at least two members.

8. A system as in claim 1 wherein said portion of said securing mechanisms used to secure said main section comprises at least four members.

9. A system as in claim 1 wherein said portion of said securing mechanisms used to secure said lower section comprises at least two members.

10. A system as in claim 1 wherein said portion of said securing mechanisms used to secure said lower section comprises at least four members.
